(19) United States
(12) Patent Application Publication
(10) Pub. No.: US 2017/0311975 A1
(43) Pub. Date: Nov. 2, 2017

(54) ELECTROSURGICAL INSTRUMENT END EFFECTOR AND SYSTEM

(71) Applicant: Gyrus Medical Limited, Cardiff (GB)

(72) Inventor: Tony Atwell, Newport (GB)

(21) Appl. No.: 15/584,337

(22) Filed: May 2, 2017

(30) Foreign Application Priority Data
May 2, 2016 (GB) 1607637.4

(51) Int. Cl. A61B 17/32; A61B 18/14; A61B 18/00; A61B 18/08

(52) U.S. Cl. CPC A61B 17/320092; A61B 18/1442; A61B 17/320016; A61B 18/082; A61B 2018/00601

(57) ABSTRACT

An electrosurgical system includes an instrument with an end effector (4) including a pair of opposing first and second jaw members (5 & 6), movable between open and closed positions. First and second electrodes (9, 17) are located on the first and second jaw members, while a third electrode (10) is also located on the first jaw member (6). The system includes first and second connections (19, 20) by which the electrodes can be connected to the output of an electrosurgical generator. The first electrode (9) is connected to the first connection (19), the second electrode (17) is connected to the second connection (20), and the third electrode (10) is connected to the first connection (19) via a capacitor (24). The third electrode (10) is more prominent than the first electrode (9) such that as the jaw members are moved to their closed position, the third electrode (10) contacts the second electrode (17) before the first electrode (9) contacts the second electrode (17).

### BACKGROUND OF THE INVENTION

This invention relates to an end effector for a surgical instrument, and to an electrosurgical system including an instrument with such an end effector. End effectors such as these are commonly used for the treatment of tissue in surgical intervention, most commonly in “keyhole” or minimally invasive surgery, but also in “open” surgery. Many different types of end effectors are known, but the present invention relates to end effectors including a pair of jaw members.

It is known to provide an electrosurgical instrument in which jaw members grasp tissue for the purpose of sealing the tissue, but there has always been difficulty in performing an effective tissue seal on vessels with widely different diameters. An instrument capable of performing an effective seal on vessels with a diameter of, say, 3 mm may not be as effective on vessels with a diameter of less than 1 mm, and vice versa. When a relatively large vessel is present within the jaws of an instrument, the resistance to compression of the tissue may prevent the jaws from contacting one another. However, when a relatively small vessel is grasped by the jaws, the tissue may have less resistance to compression such that the jaws may contact one another resulting in an electrical short between the jaws. Many jawed instruments introduce jaw stops to regulate the spacing between the jaw members when they are grasping tissue, but this still may not result in effective tissue seals being produced over a wide variety of vessel diameters.

### SUMMARY OF THE INVENTION

The present invention attempts to provide an electrosurgical system capable of performing an effective tissue seal on vessels with a relatively small diameter, as well as those having a relatively larger diameter.

Accordingly, an electrosurgical system is provided including an electrosurgical generator and an electrosurgical instrument, the electrosurgical instrument comprising

a handle including an actuation mechanism,

a pair of opposing first and second jaw members carried by the handle such that movement of the actuation mechanism causes at least one of the jaw members to move relative to the other from a first open position in which the jaw members are disposed in a spaced relation relative to one another, to a second closed position in which the jaw members cooperate to grasp tissue therebetween,

at least a first tissue-sealing electrode located on the first jaw member,

at least a second tissue-sealing electrode located on the second jaw member,

at least a third electrode located on the first jaw member, the third electrode being more prominent than the first electrode such that, as the jaw members are moved to their closed position, the third electrode contacts the second electrode before the first electrode contacts the second electrode, and

first and second connections by which the electrodes are connected to the output of the electrosurgical generator, such that coagulating RF energy is supplied to the first and second connections and hence the first and second electrodes, characterised in that

the first electrode is connected to the first connection, the second electrode is connected to the second connection, and the third electrode is connected to the first connection via a current limiting device.

The invention provides a projecting electrode which, should it come into contact with the second electrode on the opposite jaw member, still allows the instrument to seal tissue effectively. In a first situation, the third electrode is not in contact with the second electrode, and current flows between the first and third electrodes on the one hand and the second tissue-sealing electrode on the other hand. However, in a second situation the third electrode is in contact with the second electrode, and the current limiting device ensures that current flows between the first and second electrodes in preference to through the third electrode.

Preferably, the current limiting device is a capacitor. In this arrangement, the system is designed to seal either large and small diameter vessels efficiently. If a large vessel is grasped between the jaws such that both the first electrode and the third electrode are prevented by the tissue from contacting the second electrode on the opposite jaw, then both the first electrode and the third electrode are used for sealing the tissue, with current flowing from both the first electrode and the third electrode to the second electrode. However, if a relatively small vessel or small volume of tissue is grasped between the jaws, the third electrode may come into contact with the second electrode when the jaws are closed. In this circumstance, the capacitor will be driven (charged and discharged) leaving the first electrode to seal the tissue, with current flowing from the first electrode to the second electrode. The capacitor ensures that contact between the third electrode and the second electrode does not produce an excessive current flow, as would be the case from a conventional “short” between the electrodes. Preferably, the reactance of the capacitor is in the range of 0.5 microfarads to 10 microfarads.

Conveniently, the first, second and third electrodes extend longitudinally along the respective jaw members. The first, second and third electrodes preferably each have a substantially planar tissue contacting surface. In this event, the third electrode is more prominent than the first electrode such that as the jaw members are moved to their closed position the tissue contacting surface of the third electrode contacts the tissue contacting surface of the second electrode before the tissue contacting surface of the first electrode contacts the tissue contacting surface of the second electrode. The third electrode conveniently protrudes a distance of between 0.025 and 0.25 mm, preferably between 0.03 and 0.16 mm, and typically between 0.05 and 0.1 mm beyond the first electrode.

According to a convenient arrangement, the end effector includes a fourth electrode, the first third and fourth electrodes being located on the first jaw member. Typically, the third electrode is positioned between the first and fourth electrodes, preferably with the tissue contacting surfaces of the first and fourth electrodes level one with another. Conceivably, the first and fourth electrodes are formed from a unitary component having a slot therein, the third electrode being located within the slot.

The invention further resides in a method of sealing tissue comprising the steps of
i) grasping the tissue with a jawed instrument including at least first and second electrodes on one jaw of the instrument and a third electrode on the opposite jaw of the instrument,
ii) closing the jaws of the instrument so as to compress the tissue such that the first electrode comes into contact with the third electrode but the second electrode is still spaced from the third electrode,
iii) supplying coagulating RF energy between the first and second electrodes on the one hand and the third electrode on the other hand,
iv) controlling the current flowing between the first electrode and the third electrode such that it does not exceed a predetermined value even when the first electrode is in contact with the third electrode, and
v) allowing current to flow through the tissue between the second electrode and the third electrode so as to effect the sealing of the tissue.

### BRIEF DESCRIPTION OF THE DRAWINGS

The invention will now be further described, by way of example only, with reference to the accompanying drawings, in which:

FIG. 1 is a side view of an electrosurgical instrument including an end effector in accordance with the present invention, and

FIG. 2 is a schematic diagram showing the end effector of the instrument of FIG. 1, together with its connections.

### DETAILED DESCRIPTION OF EXEMPLARY EMBODIMENTS

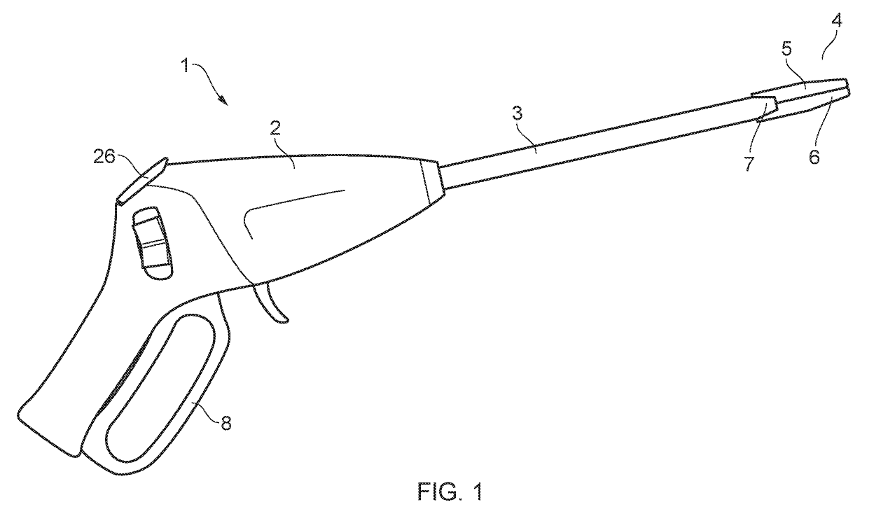

Referring to FIG. 1, an electrosurgical instrument is shown generally at 1, and comprises a handle 2 and a shaft 3 extending distally from the handle. An end effector in the form of a pair of jaws 4 is present at the distal end of the shaft. The end effector 4 comprises an upper jaw 5 and a lower jaw 6, pivotable one with respect to the other about a pivot 7. A trigger 8 present on the handle can be moved to pivot the jaws between an open position, in which the upper jaw 5 and the lower jaw 6 are spaced from each other, to a closed position (as shown in FIG. 1) in which the upper jaw 5 lies adjacent the lower jaw 6.

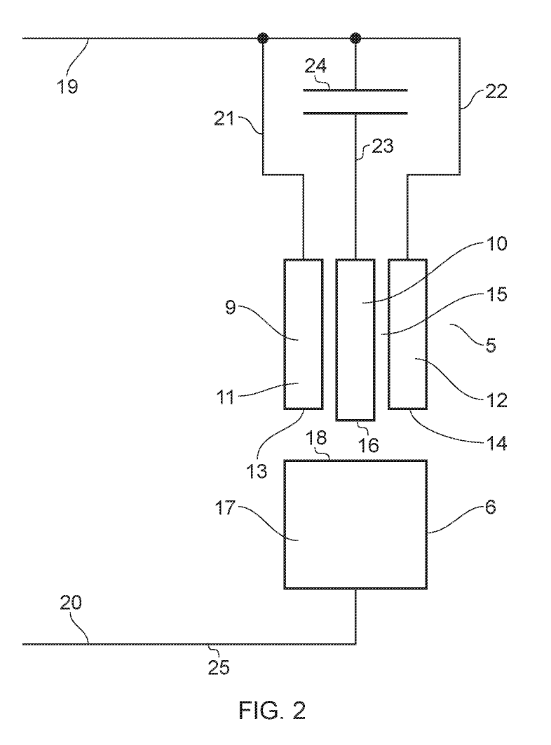

FIG. 2 shows a cross-section through the upper jaw 5 and the lower jaw 6. The upper jaw 5 has an outer electrode 9 and a central electrode 10. The outer electrode 9 is U-shaped such that it has a left portion 11 and a right portion 12. The lower surfaces of the left and right portions constitute sealing surfaces 13 & 14 respectively, the sealing surfaces being at the same height such that they are level with one another.

Between the left and right portions is a slot 15 in which the central electrode 10 is positioned. The central electrode has a greater depth such that it extends below the sealing surfaces 13 & 14. The lower surface of the central electrode constitutes a sealing surface 16, and the central electrode 10 acts independently of the electrode 9. The sealing surface 16 is below the sealing surfaces 13 and 14 by a distance of approximately 0.075 mm.

The lower jaw 6 constitutes a lower jaw electrode 17, and has a planar sealing surface 18, such that the gap at any time between the sealing surface 18 and the sealing surface 16 is less than that between the sealing surface 18 and the sealing surfaces 13 & 14. The instrument 1 also includes a first connection 19 and a second connection 20, the connections 19 & 20 being connected to opposite poles of an electrosurgical generator (not shown). Left portion 11 of the outer electrode 9 is connected to the first connection 19 by line 21, while the right portion 12 of the outer electrode 9 is connected to the first connection 19 by line 22. The central electrode 10 is connected to the first connection 19 by line 23, but between the central electrode 10 and the first connection 19 is a capacitor 24. The value of the capacitor 24 is between 0.5 and 10 nF.

Lower jaw electrode 17 is connected to the second connection 20 by means of line 25. In use, the trigger 8 is used to open and close the jaws such that tissue is grasped between the upper jaw 5 and the lower jaw 6. When tissue is firmly grasped between the jaws, a handswitch 26 on the handle is activated to provide electrosurgical energy between the connections 19 & 20. If a relatively large vessel or tissue is grasped between the jaws 5 & 6, the resistance to compression of the tissue prevents the central electrode 10 from contacting the lower jaw electrode 17. Current flows between the electrodes 9 & 10 on the one hand, and the electrode 17 on the other hand, and the tissue therebetween becomes coagulated.

If a relatively small vessel or small volume of tissue is grasped between the jaws 5 & 6, the resistance to compression may not be sufficient to prevent the central electrode 10 from contacting the lower jaw electrode 17. However, this merely causes the capacitor 24 to charge, and current still flows between the outer electrode 9 and the electrode 17. Because central electrode 10 protrudes beyond both left portion 11 and right portion 12 of outer electrode 9, the outer electrode will be prevented from contacting the lower jaw electrode 17, thereby maintaining a spacing such that shorting does not occur.

It will be appreciated by those skilled in the art that various changes can be made without departing from the scope of the present invention. For example, while electrode 9 is shown as being a unitary U-shaped electrode with left and right portions, separate left and right electrodes could be provided, connected in common to the connection 19. Those skilled in the art will appreciate that the capacitor 24 allows the central electrode 10 to come into contact with the opposite jaw, without creating an electrical short that prevents the instrument from continuing to seal tissue. As long as some part of the electrode combination remains separate from each other, current will continue to flow and the tissue will be coagulated.

What is claimed is:

1. An electrosurgical system including an electrosurgical generator and an electrosurgical instrument, the electrosurgical instrument comprising
a handle including an actuation mechanism,
a pair of opposing first and second jaw members carried by the handle such that movement of the actuation mechanism causes at least one of the jaw members to move relative to the other from a first open position in which the jaw members are disposed in a spaced relation relative to one another, to a second closed position in which the jaw members cooperate to grasp tissue therebetween,
at least a first tissue-sealing electrode located on the first jaw member,
at least a second tissue-sealing electrode located on the second jaw member,
at least a third electrode located on the first jaw member, the third electrode being more prominent than the first electrode such that, as the jaw members are moved to their closed position, the third electrode contacts the second electrode before the first electrode contacts the second electrode, and
first and second connections by which the electrodes are connected to the output of the electrosurgical generator, such that coagulating RF energy is supplied to the first and second connections and hence the first and second electrodes, characterised in that
the first electrode is connected to the first connection, the second electrode is connected to the second connection, and the third electrode is connected to the first connection via a current limiting device.

2. An electrosurgical system according to claim 1, wherein in a first situation the third electrode is not in contact with the second electrode, and current flows between the first and third electrodes on the one hand and the second tissue-sealing electrode on the other hand.

3. An electrosurgical system according to claim 1, wherein in a second situation the third electrode is in contact with the second electrode, and the current limiting device ensures that current flows between the first and second electrodes in preference to through the third electrode.

4. An electrosurgical system according to claim 1, wherein the current limiting device is a capacitor.

5. An electrosurgical system according to claim 4, wherein the reactance of the capacitor is in the range of 0.5 microfarads to 10 microfarads.

6. An electrosurgical system according to claim 1, wherein the first, second and third electrodes extend longitudinally along the respective jaw members.

7. An electrosurgical system according to claim 6, wherein the first, second and third electrodes each have a substantially planar tissue contacting surface.

8. An electrosurgical system according to claim 7, wherein the third electrode is more prominent than the first electrode such that as the jaw members are moved to their closed position the tissue contacting surface of the third electrode contacts the tissue contacting surface of the second electrode before the tissue contacting surface of the first electrode contacts the tissue contacting surface of the second electrode.

9. An electrosurgical system according to claim 8, wherein the third electrode protrudes a distance of between 0.025 and 0.25 mm beyond the first electrode.

10. An electrosurgical system according to claim 8, wherein the third electrode protrudes a distance of between 0.03 and 0.16 mm beyond the first electrode.

11. An electrosurgical system according to claim 8, wherein the third electrode protrudes a distance of between 0.05 and 0.1 mm beyond the first electrode.

12. An electrosurgical system according to claim 1, wherein the electrosurgical instrument further comprises an end effector, and wherein the end effector includes a fourth electrode, the first third and fourth electrodes being located on the first jaw member.

13. An electrosurgical system according to claim 12, wherein the third electrode is positioned between the first and fourth electrodes.

14. An electrosurgical system according to claim 13, wherein the tissue contacting surfaces of the first and fourth electrodes are level one with another.

15. An electrosurgical system according to claim 12, wherein the first and fourth electrodes are formed from a unitary component having a slot therein, the third electrode being located within the slot.

16. A method of sealing tissue comprising the steps of
i) grasping the tissue with a jawed instrument including at least first and second electrodes on one jaw of the instrument and a third electrode on the opposite jaw of the instrument,
ii) closing the jaws of the instrument so as to compress the tissue such that the first electrode comes into contact with the third electrode but the second electrode is still spaced from the third electrode,
iii) supplying coagulating RF energy between the first and second electrodes on the one hand and the third electrode on the other hand,
iv) controlling the current flowing between the first electrode and the third electrode such that it does not exceed a predetermined value even when the first electrode is in contact with the third electrode, and
v) allowing current to flow through the tissue between the second electrode and the third electrode so as to effect the sealing of the tissue.
